(19) United States
(12) Patent Application Publication
(10) Pub. No.: US 2017/0333692 A1
(43) Pub. Date: Nov. 23, 2017

(54) LEAD FOR ELECTROSTIMULATION OF A TARGET STIMULATION REGION

(71) Applicant: Boston Scientific Neuromodulation Corporation, Valencia, CA (US)

(72) Inventors: William C. Stoffregen, Lake Elmo, MN (US); Michael X. Govea, Glendale, CA (US); Bryan A. Clark, Forest Lake, MN (US)

(21) Appl. No.: 15/601,838

(22) Filed: May 22, 2017

Related U.S. Application Data
(60) Provisional application No. 62/340,426, filed on May 23, 2016.

(51) Int. Cl. A61N 1/04; A61N 1/05; A61N 1/36

(52) U.S. Cl. CPC A61N 1/04; A61N 1/3606; A61N 1/05; G02F 2201/12

(57) ABSTRACT

Various aspects of the present disclosure are directed toward apparatuses, systems and methods that include a lead for at least partially surrounding a target stimulation region. In certain instances, the lead may include a first portion, a second portion that is configured to connect with the first portion to at least partially surround the target stimulation region, and at least one electrode arranged with one of the first portion and the second portion.

### CROSS-REFERENCE TO RELATED APPLICATIONS

This application claims the benefit under 35 U.S.C. §119(e) of U.S. Provisional Patent Application Ser. No. 62/340,426, filed May 23, 2016, which is incorporated herein by reference.

### TECHNICAL FIELD

The present disclosure relates to medical devices and methods for stimulating a target region. More specifically, the disclosure relates to devices and methods that include a lead for at least partially surrounding a target stimulation region.

### BACKGROUND

Electrical stimulation may be therapeutic in a variety of diseases and disorders. Leads used in electrical stimulation may be implanted near a targeted area. In certain instances, the lead or leads may be arranged near nerves, muscles, or other tissue. The targeted areas may be difficult to locate. In addition, maintaining a location of the lead relative to the targeted areas may be difficult due to the size or location of the area.

### SUMMARY

In Example 1, a lead for at least partially surrounding a target stimulation region, the lead including: a first portion; a second portion; at least one aperture arranged with one of the first portion and the second portion an attachment element configured to connect the first portion to the second portion to at least partially surround the target stimulation region; and at least one electrode arranged with one of the first portion and the second portion.

In Example 2, the lead of Example 1, wherein the at least one electrode is arranged with the first portion, and the at least one aperture comprises a first aperture arranged with the first portion and a second aperture arranged with the second portion, the first aperture and the second aperture being configured to facilitate the attachment element connecting the first portion and the second portion.

In Example 3, the lead of Example 2, wherein the attachment element comprises a threaded element, and further comprising a threaded section arranged within the first aperture, wherein the threaded section is configured to connect with the threaded element arranged through the second aperture to connect the second portion with the first portion.

In Example 4, the lead of any of Examples 1-3, wherein an upper surface of the first portion includes a first area, and a lower surface of the second portion includes a second area, and the first area is greater than the second area.

In Example 5, the lead of Example 4, wherein the at least one electrode includes a plurality of electrodes, and the second portion covers at least a portion of the plurality of electrodes when the second portion is connected with the first portion.

In Example 6, the lead of any of Examples 1-5, further including a hinge section connecting the first portion and the second portion.

In Example 7, the lead of Example 6, wherein the first portion, the second portion, and the hinge section include an electrically insulative material.

In Example 8, the lead of Examples 6 or 7, wherein the at least one aperture comprises a first aperture arranged with the first portion and a second aperture arranged with the second portion, the hinge section is arranged at a first end of the first portion and the second portion, and the first aperture and the second aperture are arranged at a second end of the first portion and the second portion, and the first aperture and the second aperture are configured to facilitate connection between the first portion and the second portion.

In Example 9, the lead of any of Examples 1-8, wherein at least one of the first portion and the second portion includes a contoured surface.

In Example 10, the lead of Example 9, wherein the second portion includes the contoured surface, and the at least one electrode is arranged on the first portion.

In Example 11, the lead of Example 9, wherein the first portion includes the contoured surface, and the at least one electrode is arranged on the first portion, the contoured surface including a concave shape, and wherein the concave shape includes a first sloped side and a second sloped side.

In Example 12, the lead of Example 11, wherein the at least one electrode includes a plurality of electrodes, and the plurality of electrodes are arranged on the first sloped side and the second sloped side.

In Example 13, the lead of Example 12, wherein the first sloped side and the second sloped side are configured to direct the electrodes toward a central axis of the first portion.

In Example 14, the lead of any of Examples 1-13 wherein the target stimulation region includes at least one of an adrenal gland, a peripheral nerve, and tissue.

In Example 15, the lead of any of Examples 1-14, further including a pulse generator, and wherein the lead includes a lead body, and a proximal portion of the lead body is configured to connect to the pulse generator, and a distal portion of the lead body is configured to attach to at least one of the first portion and the second portion.

In Example 16, a lead for at least partially surrounding a target stimulation region, the lead including: a first portion including a first aperture; a second portion including a second aperture; an attachment element configured to connect the first portion to the second portion via the first aperture and the second aperture to at least partially surround the target stimulation region; and at least one electrode arranged with one of the first portion and the second portion.

In Example 17, the lead of Example 16, wherein the attachment element comprises a threaded element, and further comprising a threaded section arranged within the first aperture, wherein the threaded section is configured to connect with the threaded element arranged through the second aperture to connect the second portion with the first portion.

In Example 18, the lead of Example 16, wherein the attachment element comprises aa suture arranged through the first aperture and the second aperture, the suture being configured to connect the second portion with the first portion.

In Example 19, the lead of Example 18, wherein an upper surface of the first portion includes a first area, and a lower surface of the second portion includes a second area, and the first area is greater than the second area.

In Example 20, the lead of Example 19, wherein the at least one electrode includes a plurality of electrodes, and the second portion covers at least a portion of the plurality of electrodes when the second portion is connected with the first portion.

In Example 21, the lead of Example 19, wherein the first portion includes a first length and a first width, the second portion includes a second length and a second width, and at least one of the first length is approximately equal to the second length and the first width is approximately equal to the second width.

In Example 22, the lead of Example 16, wherein at least one of the first portion and the second portion includes a contoured surface.

In Example 23, the lead of Example 22, wherein the first portion includes the contoured surface, and the at least one electrode is arranged on the first portion, the contoured surface including a concave shape, and wherein the concave shape includes a first sloped side and a second sloped side.

In Example 24, the lead of Example 23, wherein the at least one electrode includes a plurality of electrodes, and the plurality of electrodes are arranged on the first sloped side and the second sloped side, and wherein the first sloped side and the second sloped side are configured to direct the electrodes toward a central axis of the first portion.

In Example 25, the lead of Example 16, further including a hinge section connecting the first portion and the second portion.

In Example 26, the lead of Example 16, wherein the target stimulation region includes at least one of an adrenal gland, a peripheral nerve, and tissue.

In Example 27, the lead of Example 16, further including a pulse generator, and wherein the lead includes a lead body, and a proximal portion of the lead body is configured to connect to the pulse generator, and a distal portion of the lead body is configured to attach to at least one of the first portion and the second portion.

In Example 28, a lead for at least partially surrounding a target stimulation region, the lead including: a first portion including a first aperture; a second portion including a second aperture; an attachment element configured to connect the first portion to the second portion via the first aperture and the second aperture to at least partially surround the target stimulation region; a hinge section connecting the first portion and the second portion; and at least one electrode arranged with one of the first portion and the second portion.

In Example 29, the lead of Example 28, wherein the first portion, the second portion, and the hinge section include an electrically insulative material.

In Example 30, the lead of Example 28, wherein the hinge section is arranged at a first end of the first portion and the second portion, and a second end of the first portion and the second portion includes the first aperture and the second aperture, and the first aperture and the second aperture are configured to facilitate connection between the first portion and the second portion.

In Example 31, the lead of Example 28, wherein at least one of the first portion and the second portion includes a contoured surface.

In Example 32, the lead of Example 28, wherein the first portion includes the contoured surface, and the at least one electrode is arranged on the first portion, the contoured surface including a concave shape, wherein the concave shape includes a first sloped side and a second sloped side, the at least one electrode includes a plurality of electrodes, and the plurality of electrodes are arranged on the first sloped side and the second sloped side.

In Example 33, a method of stimulating a target stimulation region using a lead including a first portion having a first aperture, a second portion having a second aperture, and at least one electrode arranged with one of the first portion and the second portion, the method including: delivering the first portion and the second portion of the lead to the target stimulation region; connecting the first portion to the second portion with an attachment element arranged through the first aperture and the second aperture to at least partially surround the target stimulation region; and stimulating the target stimulation region with the at least one electrode.

In Example 34, method of Example 33, wherein the attachment element comprises a threaded element, and connecting the first portion of the second portion via the first aperture and the second aperture includes arranging a threaded section within the first aperture, and arranging the threaded element through the second aperture to engage the threaded element within the threaded section to connect the second portion with the first portion.

In Example 35, method of Example 33, wherein the attachment element comprises a suture, and connecting the first portion of the second portion via the first aperture and the second aperture includes arranging the suture through the first aperture and the second aperture to connect the first portion and the second portion.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is an example illustration of a stimulation system in accordance with embodiments of the disclosure.

FIG. 2 is a top view illustration of a section of an example lead for at least partially surrounding a target stimulation region in accordance with embodiments of the disclosure.

FIG. 3A is an illustration of a section of another example lead for at least partially surrounding a target stimulation region in accordance with embodiments of the disclosure.

FIG. 3B is a side view of the example lead shown in FIG. 3A in accordance with embodiments of the disclosure.

FIG. 3C is a top view of the example lead shown in FIGS. 3A-B in accordance with embodiments of the disclosure.

FIG. 4A is an illustration of a section of another example lead for at least partially surrounding a target stimulation region in accordance with embodiments of the disclosure.

FIG. 4B is a side view of the example lead shown in FIG. 4A in accordance with embodiments of the disclosure.

FIG. 4C is a top view of the example lead shown in FIGS. 4A-B in accordance with embodiments of the disclosure.

FIG. 5A is an illustration of a first portion and a second portion of an example lead for at least partially surrounding a target stimulation region in accordance with embodiments of the disclosure.

FIG. 5B is an end view of the first portion and the second portion shown in FIG. 5A in accordance with embodiments of the disclosure.

FIG. 6A is an illustration of a section of another example lead for at least partially surrounding a target stimulation region in accordance with embodiments of the disclosure.

FIG. 6B is a side view of the example lead shown in FIG. 6A in accordance with embodiments of the disclosure.

FIG. 7 is an illustration of an example lead that includes a clamp attachment mechanism for at least partially surrounding a target stimulation region in accordance with embodiments of the disclosure.

FIG. 8 is an illustration of another example lead that includes attachment mechanisms for at least partially surrounding a target stimulation region in accordance with embodiments of the disclosure.

FIG. 9A is an illustration of an example deployment tool and example portion of a lead for at least partially surrounding a target stimulation region in accordance with embodiments of the disclosure.

FIG. 9B is an illustration of the portion of the lead, as shown in FIG. 9B, deployed from the deployment tool in accordance with embodiments of the disclosure.

FIG. 10 is a flow chart of an example method of stimulating a target stimulation region using a lead that includes a first portion and a second portion in accordance with embodiments of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

As the terms are used herein with respect to ranges of measurements (such as those disclosed immediately above), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

Although the term “block” may be used herein to connote different elements illustratively employed, the term should not be interpreted as implying any requirement of, or particular order among or between, various steps disclosed herein unless and except when explicitly referring to the order of individual steps.

### DETAILED DESCRIPTION

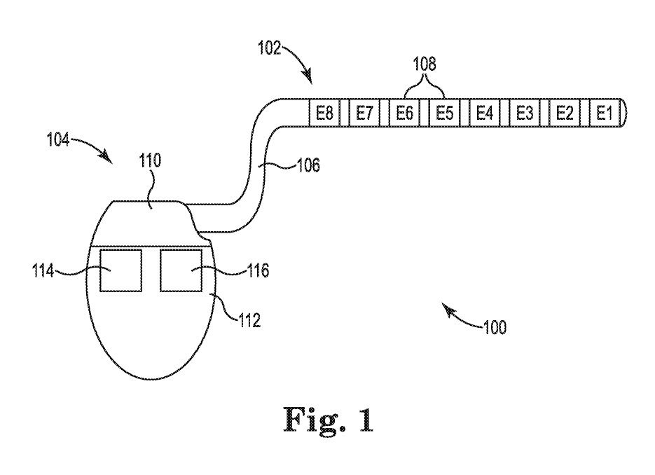

FIG. 1 is an example illustration of a stimulation system 100, which includes a lead 102 and a controller 104 in accordance with embodiments of the disclosure. The lead 102 may include an elongated cylindrical lead body 106. The lead 102 includes a number of electrodes 108 arranged on the lead body 106. The electrodes 108 may be arranged circumferentially around the lead 102 as ring electrodes mounted to the lead body 106. Each of the plurality of electrodes 108 are labeled E1-E8, however the actual number and shape of leads and electrodes may vary according to the application.

As shown, a proximal portion of the lead body 106 communicatively couples the lead 102 to the controller 104. A connector 110 arranged with the controller 104 couples an end of the lead 102 to the controller 104, thereby communicatively (e.g., electrically) coupling the electrodes 108 to the internal electronics within the controller 104. In embodiments, the lead 102 may be wirelessly coupled to the controller 104, which may be configured to control one or more remote leads 102. That is, for example, the lead body 106 may include an energy source such as an a transducer, a coil, a capacitor, and/or the like, which may be configured to provide energy to the electrodes 108 in response to receiving a wirelessly-transmitted instruction from the controller 104

The controller 104 may also include a housing 112, which contains and houses electronic and other components. In embodiments, the controller 104 may include a pulse generator that may be implantable within a patient (e.g., an implantable pulse generator (IPG)), or configured to be positioned external to the patient. In instances where the controller 104 is implantable, the housing 112 may be formed of an electrically conductive, biocompatible material, such as titanium, and may form a hermetically sealed compartment wherein the internal electronics are protected from the body tissue and fluids.

The housing 112 may enclose sensing circuitry 114 configured to receive, from one or more of the electrodes 108, physiological signals obtained by the one or more electrodes 108. The housing 112 may also enclose pulse generation circuitry 116 that delivers stimulation energy via one or more of the electrodes 108. According to various embodiments, the sensing circuitry 114 (or aspects thereof) and/or the pulse generation circuitry 116 (or aspects thereof) may be configured to be implanted in the patient and/or disposed external to the patient. That is, for example, in embodiments, the sensing circuitry 114 and the pulse generation circuitry 116 may be integrated within a processor disposed in an implantable medical device (e.g., the controller 104) and/or an external medical device. The sensing circuitry 114 (or aspects thereof) and/or the pulse generation circuitry 116 (or aspects thereof) may be implemented in any combination of hardware, firmware, and software. For example, the sensing circuitry 114 may be, or include, a first algorithm, virtual processor, and/or process implemented by a processor, and, similarly, the pulse generation circuitry 116 may be, or include, a second algorithm, virtual processor, and/or process implemented by a processor. In embodiments, the sensing circuitry 114 may be, or include, a first set of physical and/or virtual circuit elements, and, similarly, the pulse generation circuitry 116 may be, or include, a second set of physical and/or virtual circuit elements.

In embodiments, the controller 104 may include a programmable micro-controller or microprocessor, and may include one or more programmable logic devices (PLDs) or application specific integrated circuits (ASICs). In some implementations, the controller 104 may include memory as well. Although embodiments of the present system 100 are described in conjunction with a controller 104 having a microprocessor-based architecture, it will be understood that the controller 104 (or other device) may be implemented in any logic-based integrated circuit architecture, if desired. The controller 104 may include digital-to-analog (D/A) converters, analog-to-digital (ND) converters, timers, counters, filters, switches, and/or the like.

The stimulation energy may be in the form of a pulsed electrical waveform delivered to tissue via one or more of the electrodes 108 in accordance with a set of stimulation parameters, which may be programmed into the controller 104, transmitted to the controller 104, and/or the like. Stimulation parameters may include, for example, electrode combinations that define which electrodes are activated as anodes (positive), which electrodes are activated as cathodes (negative), and which electrodes are not activated; percentages of a total amount of stimulation energy assigned to certain electrodes (fractionalized electrode configurations); and/or electrical pulse parameters, which may define the pulse amplitude (measured in milliamps or volts depending on whether the controller 104 supplies constant current or constant voltage to one or more of the electrodes 108), pulse duration (measured in microseconds), pulse rate (measured in pulses per second), and/or burst rate. The pulse generation circuitry 116 may be capable of providing the stimulation energy to the one or more of the electrodes 108 over multiple channels or over only a single channel. Stimulation energy may be used to identify therapy regions and/or to provide stimulation therapy to identified therapy regions. In embodiments, stimulation energy may be used in conjunction with treatment substrates, as described herein.

In addition, the stimulation energy may be transmitted to a target stimulation region in a monopolar or multipolar (e.g., bipolar, tripolar, etc.) fashion. In certain instances, the pulse generation circuitry 116 may individually control the magnitude of electrical current flowing through each of the electrodes. In these instances, current generators may be used to supply current-regulated amplitudes to selectively generate independent current sources for one or more of the electrodes 108.

The sensing circuitry 114 may be configured to receive a physiological signal obtained by one or more of the electrodes 108, and analyze the received physiological signal to identify a target stimulation region. According to embodiments, the physiological signal may include intrinsic electrical activity, a physiological response to an applied stimulation signal, and/or the like. For example, the sensing circuitry 114 may be configured to obtain a physiological signal that is a response to a stimulation signal administered using one or more of the electrodes 108, and to analyze that signal to identify a target stimulation region.

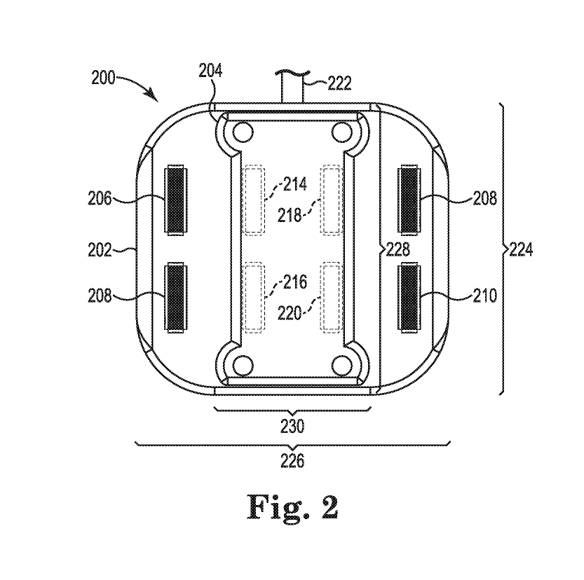

FIG. 2 is a top view illustration of a section of an example lead 200 for at least partially surrounding a target stimulation region in accordance with embodiments of the disclosure. The lead 200 may include a first portion 202, a second portion 204, and at least one electrode arranged with one of the first portion 202 and the second portion 204. The second portion 204 is configured to connect with the first potion 202 to at least partially surround the target stimulation region. In certain instances, it may be beneficial for the lead 200 to be held in one place to at least partially surround the target stimulation region. Such target stimulation regions, for example, may be an adrenal gland, a peripheral nerve, tissue, or other similar regions that may be difficult to locate and/or difficult to provide targeted stimulation due to the location and/or size of the target region. In addition, the target stimulation region may change location due to movement of a patient. Thus, the first portion 202 and the second portion 204 may connect to at least partially surround the adrenal gland, peripheral nerve, and/or tissue to allow for targeted simulation of the target stimulation. More specifically and in certain instances, it may be desirable to provide intermittent or continuous stimulation to the target stimulation region. The first portion 202 and the second portion 204 may hold at least a portion of the target stimulation region therebetween to provide stimulation to the target stimulation region. Thus, the first portion 202 and the second portion 204 may provide an anchor-like arrangement to hold the lead 200 in the desired location.

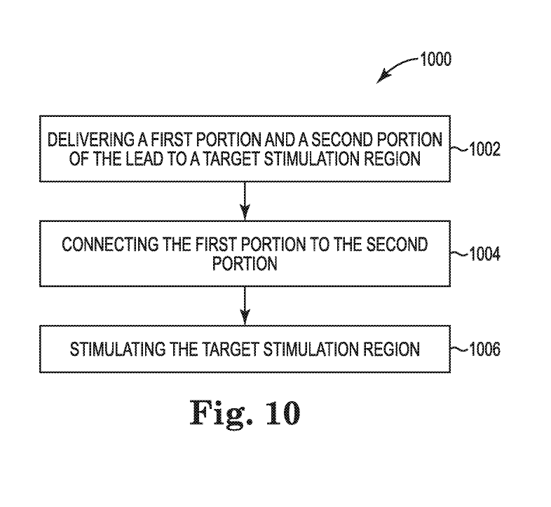

In certain instances, the first potion 202 and the second portion 204 are separate pieces, which may be connected after manufacturing. In certain instances, the first potion 202 and the second portion 204 are configured to connect as part of procedure to deliver the lead 200 to the target stimulation region or prior to the procedure to deliver the lead 200 to the target stimulation region (e.g., as described with reference to FIG. 10).

As shown in FIG. 2, the lead 200 includes a plurality of electrodes 206-210 (although the lead 200 may include one, two, three, four, five, six, seven, or eight or more electrodes). To provide stimulation energy to the target stimulation region, the plurality of electrodes 306-320 may be communicatively and/or electrically coupled to a controller, such as the controller 104 described above with reference to FIG. 1, which may include a pulse generator. The lead 200 may be electrically coupled to the controller by a leady body 222. The lead body 222 may also be coupled to one of the first portion 202 and the second portion 204. As shown in FIG. 2, the lead body 222 is coupled to the first portion 202 and the plurality of electrodes 206-220 is arranged thereon. In certain instances, the lead body 222 may be configured to attach to one of the first portion 202 and the second portion 204.

In addition, the plurality of electrodes 206-220 are shown arranged with the first portion 202, however, the plurality of electrodes 206-220 may be arranged with the second portion 204, or some of the plurality of electrodes 206-220 may be arranged with the first portion 202 and others of the plurality of electrodes 206-220 may be arranged with the second portion 204. In certain instances, and as is shown in FIG. 2, the first portion 202 includes a first area, and the second portion 204 includes a second area with the first area being greater than the second area. As a result, some or all of the plurality of electrodes 206-220 may be covered when the first portion 202 is connected to the second portion 204. As shown in FIG. 2, the second portion 204 covers electrodes 214-220 (shown in phantom), and other electrodes 206-210 are not covered. In certain instances, the first portion 202 includes a first length 224 and a first width 226, the second portion 204 includes a second length 228 and a second width 230. As is shown in FIG. 2, the first length 224 is approximately equal to the second length 228, and the first width 226 is greater than the second width 230.

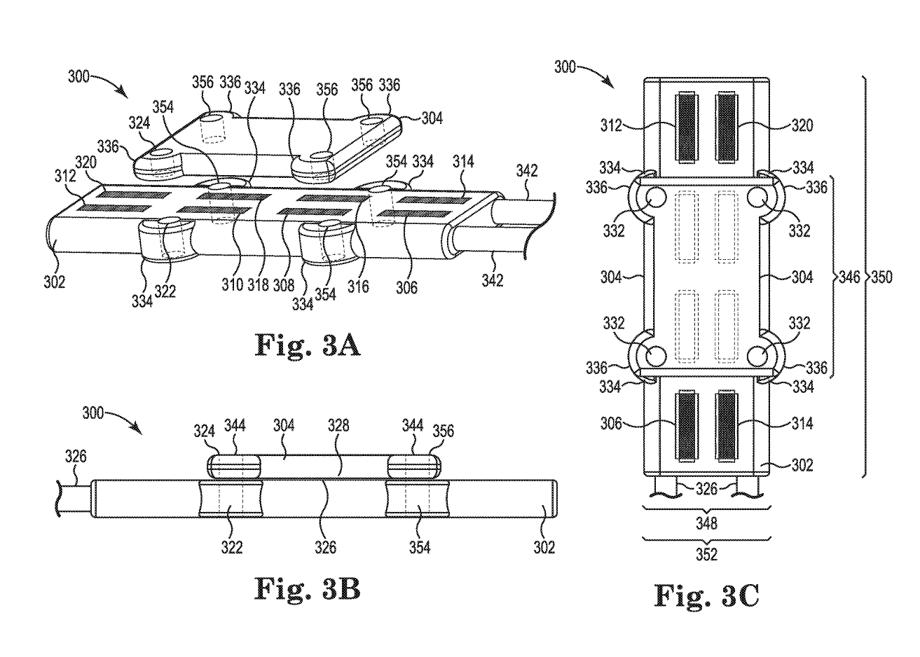

FIG. 3A is an illustration of a section of an example lead 300 for at least partially surrounding a target stimulation region in accordance with embodiments of the disclosure. The lead 300 may include a first portion 302, a second portion 304, and at least one electrode arranged with one of the first portion 302 and the second portion 304. Although the lead 300 in FIG. 3A includes a plurality of electrodes 306-320, the lead 300 may include between one, two, three, four, five, six, seven, or eight or more electrodes. In addition, the plurality of electrodes 306-320 are shown arranged with the first portion 302, however, the plurality of electrodes 306-320 may be arranged with the second portion 304, or some of the plurality of electrodes 306-320 may be arranged with the first portion 302 and others of the plurality of electrodes 306-320 may be arranged with the second portion 304.

In addition, the first portion 302 and the second portion 304 may be configured to connect to one another to at least partially surround the target stimulation region. In certain instances, it may be beneficial for the lead 300 to be held in one place and/or at least partially surround a target stimulation region. In other instances, precise and/or targeted simulation of the target stimulation region may be beneficial. The target stimulation region may be difficult to locate, or the target stimulation region may change location due to movement of a patient. Such target stimulation regions, for example, may be an adrenal gland, a peripheral nerve, and tissue. Thus, the first portion 302 and the second portion 304 may connect to at least partially surround the adrenal gland, peripheral nerve, and/or tissue to allow for targeted simulation of the target stimulation using one or more of the plurality of electrodes 306-320.

To provide stimulation energy to the target stimulation region, the plurality of electrodes 306-320 may be communicatively and/or electrically coupled to a controller, such as the controller 104 described above with reference to FIG. 1, which may include a pulse generator. The lead 300 may be wirelessly coupled to the controller, or may be electrically coupled to the controller by a lead body 342. The lead body 342 may also be coupled to one of the first portion 302 and the second portion 304. As shown in FIG. 3A, the lead body 342 is coupled to the first portion 302 and the plurality of electrodes 306-320 arranged thereon. In certain instances, the lead body 342 may be configured to attach to one of the first portion 302 and the second portion 304.

The first portion 302 and the second portion 304 may also include, respectively, a first aperture 322 and a second aperture 324. The first portion 302 and the second portion 304 may include additional first portion apertures 354 and additional second portion apertures 356 as shown in FIG. 3A. The first aperture 322 and the second aperture 324 may be configured to facilitate connection between the first portion 302 and the second portion 304. As is discussed in further detail below with reference to FIG. 3C, the first aperture 322, the second aperture 324, the additional first portion apertures 354 and the additional second portion apertures 356 may be arranged, respectively, through protrusion sections 334, 336 of the first portion 302 and the second portion 304.

FIG. 3B is a side view of the example lead 300 shown in FIG. 3A in accordance with embodiments of the disclosure. As shown in FIG. 3B, the first aperture 322 and the second aperture 324 may be counterparts of one another. Similarly, the additional first portion apertures 354 and the additional second portion apertures 356 may be counterparts of one another. Thus, the first aperture 322 and the second aperture 324 may form a pathway 344 through which an attachment element may be provided to connect and hold together the first portion 302 and the second portion 304. The additional first portion apertures 354 and the additional second portion apertures 356 may also form a pathway 344 through which the attachment element may be provided to connect and hold together the first portion 302 and the second portion 304. In addition, the attachment element (not shown) may have a similar shape and size as each of the first aperture 322 and the second aperture 324 and therefore provide a friction fit connection within each of the first aperture 322 and the second aperture 324 to connect the first portion 302 and the second portion 304. The attachment element may be provided to connect and hold together the first portion 302 and the second portion 304 through the first aperture 322 and the corresponding second aperture 324, and/or through any one or more of the additional first portion apertures 354 and the corresponding additional second portion apertures 356. The attachment element may be arranged through the first aperture 322 and the second aperture 324 (and/or through any one or more of the additional first portion apertures 354 and the corresponding additional second portion apertures 356) subsequent to arranging the first portion 302 and the second portion 304 adjacent one another. In certain instances, the first portion 302 and the second portion 304 may be connected together during a laparoscopic procedure or other surgical procedure. More specifically, the first portion 302 and the second portion 304 may be separate when delivered to the target stimulation region. Thus, the first portion 302 and the second portion 304 may be connected to at least partially surround the target stimulation region as part of that procedure. In addition, the attachment element may be arranged with one of the first aperture 322 and the second aperture 324 prior to arranging the first portion 302 and the second portion 304 adjacent one another.

In certain instances, the attachment element may be a threaded element (shown in FIG. 3C). The first aperture 322 may include a corresponding threaded section that is configured to connect with the threaded element when the attachment element is arranged through the second aperture 324 to connect the second portion 304 with the first portion 302. The threaded section may be formed by the first aperture 322 itself, or by an additional threaded material. The attachment element may be a suture arranged through the pathway 344 formed by the first aperture 322 and the second aperture 324. The suture may configured to connect the second portion 304 with the first portion 302 as part of a procedure to deliver the lead 300 to the target stimulation region (e.g., as described with reference to FIG. 11).

As is also shown in FIG. 3B, the plurality of electrodes 306-320 are mounted flush with an upper surface 326 of the first portion 302. In addition, a space is formed between the upper surface 326 of the first portion 302 and a lower surface 328 of the second portion 304. As used herein, the terms “upper surface” and “lower surface” are used to refer to certain features, but are characterized in the context of the illustrations for clarity and to describe relative orientations of features with respect to other features, and are not intended to imply any particular orientation of a lead, or absolute (or preferred) orientations of features thereof. The connection between the first portion 302 and the second portion 304 may be varied in order for the space between the upper surface 326 of the first portion 302 and the lower surface 328 of the second portion 304 to accommodate the target stimulation region. As a result of the plurality of electrodes 306-320 being mounted flush with the upper surface 326 of the first portion 302, the target stimulation region may be adjacent the plurality of electrodes 306-320.

FIG. 3C is a top view of the example lead 300 shown in FIGS. 3A-B in accordance with embodiments of the disclosure. The first portion 302 and the second portion 304 are shown connected together via the threaded element 332. The threaded element 332 may be formed of a metal screw-like element to connect the first portion 302 and the second portion 304.

As shown in FIG. 3C (and FIG. 3A), the first aperture 322, the second aperture 324, the additional first portion apertures 354 and the additional second portion apertures 356 may be arranged, respectively, through protrusion sections 334, 336 of the first portion 302 and the second portion 304. The protrusion sections 334, 336 are formed on the perimeter of each of the first portion 302 and the second portion 304. The protrusion sections 334, 336 may allow for the plurality of electrodes 306-320 to be uniformly mounted in an array on the first portion 302 without interfering with the operation of the plurality of electrodes 306-320.

In certain instances and as is shown in FIG. 3C, the first portion 302 has a first area, and the second portion 304 has a second area with the first area being greater than the second area. As a result, some or all of the plurality of electrodes 306-320 may be covered when the first portion 302 is connected to the second portion 304. As shown in FIG. 3C, the second portion 304 covers electrodes 308, 310, 316, 318 (shown in phantom), and other electrodes 306, 312, 314, 320 are not covered. In certain instances, the first portion 302 includes a first length 346 and a first width 348, the second portion 304 includes a second length 350 and a second width 352. As shown in FIG. 3C, the first width 348 may be approximately equal to the second width 352, and the first length 346 is less than the second length 350. Although the first and second portions 302 and 304 are illustrated as having a generally rectangular shape, embodiments may include first and/or second portions having other shapes such as, for example, oblong shapes, elliptical shapes, other curved shapes, and/or the like.

The illustrative components shown in FIGS. 3A-C are not intended to suggest any limitation as to the scope of use or functionality of embodiments of the disclosed subject matter. Neither should the illustrative components be interpreted as having any dependency or requirement related to any single component or combination of components illustrated therein. Additionally, any one or more of the components depicted in any of the FIGS. 1, 2, and 4-6 may be, in embodiments, integrated with various other components depicted therein (and/or components not illustrated), all of which are considered to be within the ambit of the disclosed subject matter. For example, the lead 300 described with reference to FIGS. 3A-C may be used in the system 100. More specifically, the section of the lead 200 shown in FIGS. 3A-C may be used in place of the electrodes 108 shown in FIG. 1. In addition, the system 100 and lead 300 shown in FIGS. 3A-C may be used in connection with embodiments of the method described with reference to FIG. 10. Further and as shown in FIGS. 3A-C, the lead body 342 may include two components. In these instances, the lead body 342 may split from a single lead (e.g., as shown in FIG. 1) at some point near a distal end of the lead body 342 into two components. In other instances, the lead 300 may include a lead body having a single component (e.g., as shown in FIG. 1), which may be connected or fused to one of the first portion 302 and the second portion 304.

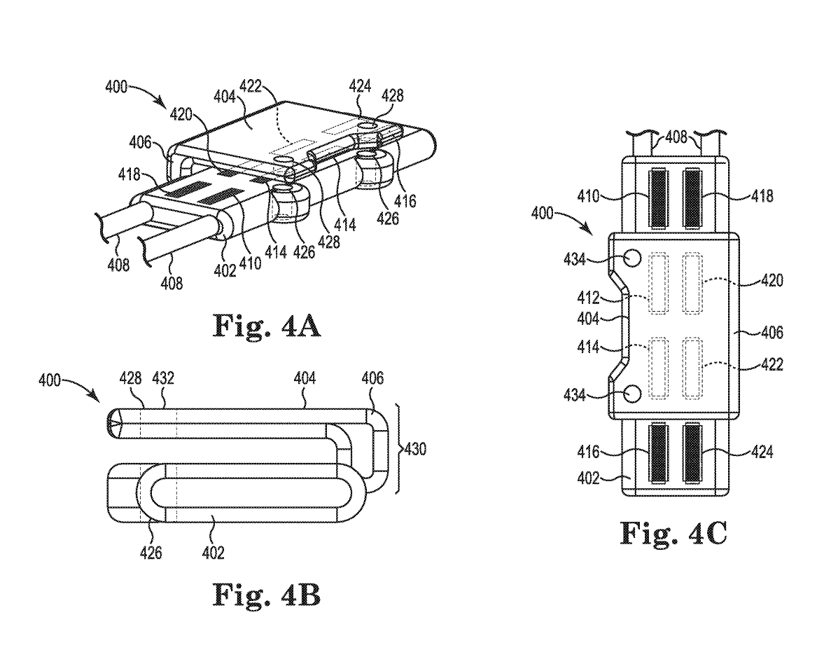

FIG. 4A is an illustration of a section of another example lead 400 for at least partially surrounding a target stimulation region in accordance with embodiments of the disclosure. The lead 400 may include a first portion 402, a second portion 404, and a hinge section 406. The second portion 404 is configured to connect with the first potion 402 to at least partially surround the target stimulation region. In addition, one or more of the first portion 402 and the second portion 404 may include at least one electrode. The hinge section 406 may be formed between, and moveably connect, the first portion 402 and the second portion 404. As shown, the hinge section 406 is arranged along a length of the first portion 402 and the second portion 404. In certain instances, the hinge section 406 may be arranged along a width of the first portion 402 and the second portion 404.

In certain instances, the first portion 402 also includes first apertures 426 and the second portion 404 includes second apertures 428. The first apertures 426 and the second apertures 428 may be configured to facilitate connection between the first portion 402 and the second portion 404. The hinge section 406 may be arranged at a first end of the second portion 404, and the first apertures 426 and the second apertures 428 may be arranged at or near a second (an opposing) end of the first portion 402 and the second portion 404. The first apertures 426 and the second apertures 428 may be counterparts of one another. An attachment element may be provided through the first apertures 426 and the second apertures 428 to connect and hold together the first portion 402 and the second portion 404. In addition, the attachment element (not shown) may have a similar shape and size as each of the first apertures 426 and the second apertures 428 and therefore provide a friction fit connection within each of the first apertures 426 and the second apertures 428 to connect the first portion 402 and the second portion 404. The first apertures 426 and the second apertures 428 may serve as conduits for connections in addition to the connection provided by the hinge section 406. The hinge section 406 may be arranged to partially surround the target stimulation region. In certain instances, the first portion 402 and the second portion 404 will be arranged on either side of the target stimulation region such that the target stimulation region is between the first portion 402 and the second portion 404. Subsequent to the hinge section 406 being arranged to partially surround the target stimulation region, the attachment element may be arranged through the first apertures 426 and the second apertures 428 to connect the first portion 402 and the second portion 404.

In addition and as is shown in FIG. 4, the lead 400 includes a plurality of electrodes 410-424 (although the lead 400 may include one, two, three, four, five, six, seven, or eight or more electrodes). To provide stimulation energy to the target stimulation region, the plurality of electrodes 410-424 may be communicatively and/or electrically coupled to a controller, such as the controller 104 described above with reference to FIG. 1, which may include a pulse generator. The lead 400 may be electrically coupled to the controller by a lead body 408. The lead body 408 may also be coupled to one of the first portion 402 and the second portion 404. As shown in FIG. 4, the lead body 408 is coupled to the first portion 402 and the plurality of electrodes 410-424 arranged thereon.

FIG. 4B is a side view of the example lead 400 shown in FIG. 4A in accordance with embodiments of the disclosure. The hinge section 406 includes a height 430 configured such that the target stimulation region may fit between the first portion 402 and the second portion 404. An attachment element (e.g., a suture or threaded element 434 shown in FIG. 4C) may be arranged through a pathway 432 to further connect the first portion 402 and the second portion 404. In certain instances, the attachment element may be arranged through the pathway 432 and may be configured to be adjusted. The connection of the second portion 404 and the first portion 402 through the first apertures 426 and the second apertures 428 may be tightened to a point where the second portion 404 may be angled from the hinge section 406 toward the first apertures 426 and the second apertures 428.

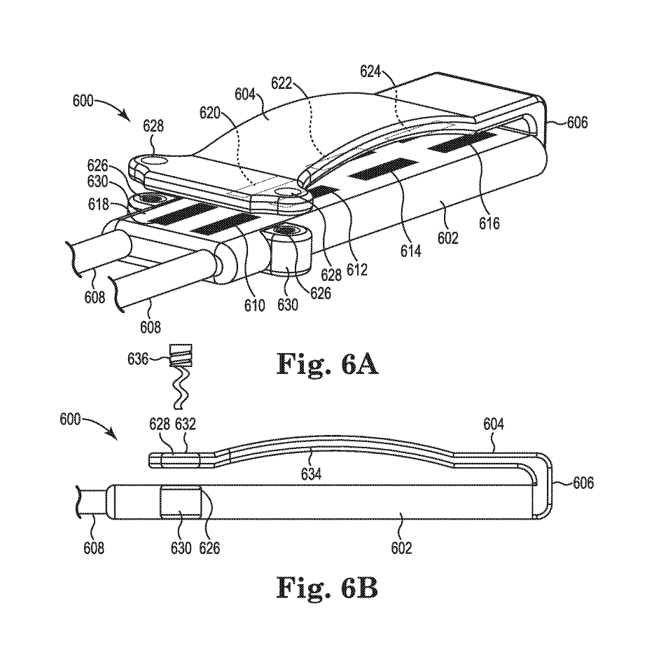

FIG. 4C is a top view of the example lead 400 shown in FIGS. 4A-B in accordance with embodiments of the disclosure. In certain instances, the attachment element arranged through the first apertures 426 and the second apertures 428 may be a threaded element 434. In addition, the first apertures 426 may include a corresponding threaded section is configured to connect with the threaded element 434 when arranged through the second apertures 428 to connect the second portion 404 with the first portion 402. The threaded section may be formed by the first apertures 426 itself, or by an additional threaded material (e.g., as is shown in FIG. 6B).

As is shown in FIG. 4C, the first portion 402 includes a first area, and the second portion 404 includes a second area with the first area being greater than the second area. As a result, some or all of the plurality of electrodes 410-424 may be covered when the first portion 402 is connected to the second portion 404. As shown in FIG. 4C, the second portion 404 covers electrodes 412, 414, 420, 422 (shown in phantom), and other electrodes 410, 416, 418, 424 are not covered.

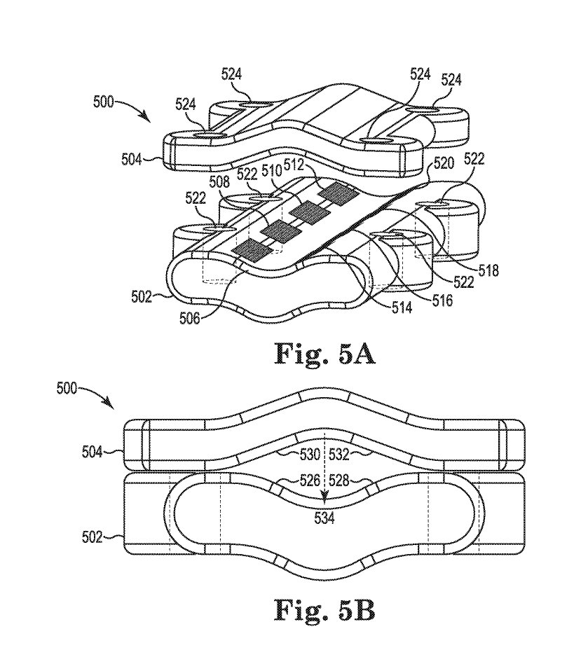

FIG. 5A is an illustration of a first portion 502 and a second portion 504 of an example lead 500 for at least partially surrounding a target stimulation region in accordance with embodiments of the disclosure. The lead 500 may include a first portion 502, a second portion 504, and a plurality of electrodes 506-520 arranged with the first portion 502. The second portion 504 is configured to connect with the first potion 502 to at least partially surround the target stimulation region (e.g., an adrenal gland, a peripheral nerve, and tissue of a patient). In certain instances, the first potion 502 and the second portion 504 are configured to connect as part of procedure to deliver the lead 500 to the target stimulation region or prior to the procedure to deliver the lead 500 to the target stimulation region (e.g., as described with reference to FIG. 10). To provide stimulation energy to the target stimulation region, the plurality of electrodes 506-520 may be communicatively and/or electrically coupled to a controller, such as the controller 104 described above with reference to FIG. 1, which may include a pulse generator.

The first portion 502 and the second portion 504 may also include, respectively, first apertures 522 and second apertures 524. In certain instances, first apertures 522 and the second apertures 524 may be connected using an attachment element such as a threaded element (shown in FIG. 3C), a suture, pin, clip, or any other type of attachment mechanism that may be arranged through the first apertures 522 and the second apertures 524.

FIG. 5B is an end view of the first portion 502 and the second portion 504 shown in FIG. 5A in accordance with embodiments of the disclosure. In certain instances, the at least one of the first portion 502 and the second portion 504 include a contoured surface, and as is shown in FIG. 5B, the first portion 502 and the second portion 504 include contoured surfaces. Each contoured surface may be a concave shape. More specifically, the first portion 502 includes a first sloped surface 526 and a second sloped surface 528, and the second portion 504 includes a first sloped surface 530 and a second sloped surface 532. The plurality of electrodes 506-520 may be arranged on any of the sloped surfaces. As is shown in FIG. 5B, the plurality of electrodes 506-520 are arranged on each of the first sloped surface 526 and the second sloped surface 528 of the first portion 502. The first sloped surface 526 and the second sloped surface 528 may have shape such that each of the plurality of electrodes 506-520 is directed toward a central axis 534 of the first portion 502. The target stimulation region may be arranged therein. In addition, the first portion 502 and the second portion 504 include approximately equal areas such that each of the plurality of electrodes 506-520 are covered when the first portion 502 and the second portion 504 are connected.

FIG. 6A is an illustration of a section of another example lead 600 for at least partially surrounding a target stimulation region in accordance with embodiments of the disclosure. The lead 600 may include a first portion 602, a second portion 604, and a hinge section 606. The lead 600 may also include a lead body 608. The lead body 608 may be electrically coupled to a plurality of electrodes 610-624 that are arranged with the first portion 602. The lead body 608 may also communicatively and/or electrically couple the plurality of electrodes 610-624 to a controller, such as the controller 104 described above with reference to FIG. 1, which may include a pulse generator.

The hinge section 606 may be formed between and connect the first portion 602 and the second portion 604. As shown, the hinge section 606 is arranged along a width of the first portion 602 and the second portion 604. In certain instances, the first portion 602 and the second portion 604 also, respectively, include first apertures 626 and second apertures 628. The first apertures 626 and the second apertures 628 may be configured to facilitate connection between the first portion 602 and the second portion 604. The hinge section 606 may be arranged at a first end of the first portion 602 and the second portion 604, and the first apertures 626 and the second apertures 628 may be arranged at a second (an opposing) end of the first portion 602 and the second portion 604. In addition, an attachment element may be provided through the first apertures 626 and the second apertures 628 to connect and hold together the first portion 602 and the second portion 604. The hinge section 606 may be arranged to partially surround the target stimulation region. In certain instances, the first portion 602 and the second portion 604 will be arranged on either side of the target stimulation region such that the target stimulation region is between the first portion 602 and the second portion 604. Subsequent to the hinge section 606 being arranged to partially surround the target stimulation region, the attachment element may be arranged through the first apertures 626 and the second apertures 628 to connect the first portion 602 and the second portion 604 and hold the lead 600 in place. As is shown in FIG. 6B, the second apertures 628 may include a threaded section 630 arranged therethrough.

FIG. 6B is a side view of the example lead 600 shown in FIG. 6A in accordance with embodiments of the disclosure. An attachment element may be arranged through a pathway 632 formed by the first apertures 626 and the second apertures 628 to further connect the first portion 602 and the second portion 604. As shown in FIG. 6B, the component may be a threaded element 636. As is shown in FIG. 6A, the second apertures 628 includes the threaded section 630 arranged therethrough. The threaded element 636 may be arranged through the pathway 632 and threaded into the threaded section 630 to connect the first portion 602 and the second portion 604. As shown, the threaded element 636 may be formed of a metal screw-like element to connect the first portion 602 and the second portion 604. The threaded element 636 and the threaded section 630 may be similarly sized to facilitate connection between the threaded element 636 and the threaded section 630.

In certain instances, the second portion 604 may include a contoured surface 634. The contoured surface 634 may be concave, and may assist in direct electrical stimulation energy, provided from one or more of the plurality of electrodes 610-624, to the target stimulation region.

The illustrative components shown in FIGS. 5 and 6 are not intended to suggest any limitation as to the scope of use or functionality of embodiments of the disclosed subject matter. Neither should the illustrative components be interpreted as having any dependency or requirement related to any single component or combination of components illustrated therein. Additionally, any one or more of the components depicted in any of FIGS. 1-4 may be, in embodiments, integrated with various other components depicted therein (and/or components not illustrated), all of which are considered to be within the ambit of the disclosed subject matter. For example, the leads 500 and 600 may be used in the system 100. More specifically, the section of the leads 500 and 600 shown in FIGS. 5-6 may be used in place of the electrodes 108 shown in FIG. 1. In addition, the system 100 and leads 500 and 600 shown in FIGS. 5-6 may be used in connection with embodiments of the method described with reference to FIG. 10. Further, the leads 200, 300, and 400 described with reference to FIGS. 1-4 may include contoured surfaces as shown in FIGS. 5-6.

The lead portions (e.g., first portions 202, 302, 402, 502, and 602; and second portions 204, 304, 404, 504, and 604) described herein may be formed of an insulative material. In instances where one or more of the lead portions include one or more electrodes, the insulative material may serve to insulate individual electrodes from one another. Example insulative materials that may be used in the manufacture of the various lead portions described herein include silicone, polyurethanes (PU), polytetrafluroethylene (PTFE), ethylene tetrafuloroethylene (ETFE), polyisobutylene polyurethane (PIB-PUR), polyether ether ketone (PEEK), poly(styrene-block-isobutylene-block-styrene) (SIBS), or any suitable biocompatible insulative material.

In additional, the electrodes discussed herein may include materials such as platinum, platinum iridium, titanium, stainless steel, or any suitable biocompatible conductive material. The electrodes discussed herein may further be coated with a second conductive material exhibiting beneficial chemical, electrochemical, or physical properties such as iridium, iridium oxide, or titanium nitride. The attachment of electrodes to lead portions (e.g., first portions 202, 302, 402, 502, and 602; and second portions 204, 304, 404, 504, and 604) may be accomplished by mechanically fixing the electrode into the lead portions, which may be formed of insulative material, via an overmolding process. The attachment of electrodes to lead portions may also occur during a bonding process and/or the electrodes may be chemically bonded to the lead portions. When chemically bonded, a primer layer may be applied to the electrodes prior to molding or bonding.

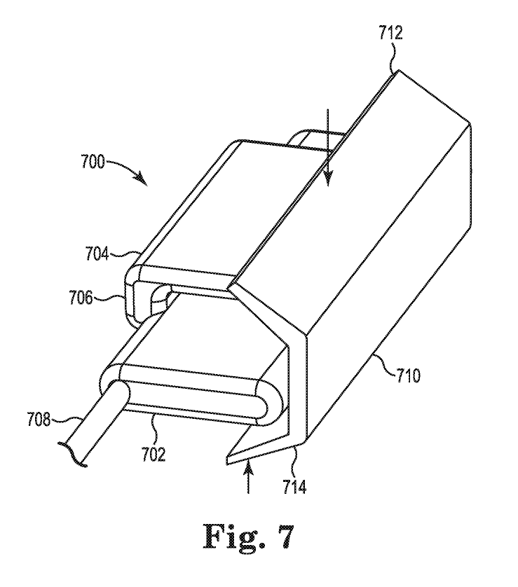

FIG. 7 is an illustration of an example lead 700 that includes a clamp attachment mechanism 712 for at least partially surrounding a target stimulation region in accordance with embodiments of the disclosure. The lead 700 may include a first portion 702, a second portion 704, and a hinge section 706. The lead 700 may also include a lead body 708. As described in further detail above, the lead body 608 may be electrically coupled to at least one electrode (not shown) arranged with the first portion 702 or the second portion 704. The lead 700 may be configured to at least partially surround the target stimulation region by connecting together the first portion 702 and the second portion 704.

As shown, the clamp attachment mechanism 712 at least partially surrounds the first portion 702 and the second portion 704. A force (represented by the arrows shown) may be applied to the clamp attachment mechanism 712 to hold together the first portion 702 and the second portion 704. The force may be applied by a surgical tool such as a forceps or the like.

The illustrative components shown in FIG. 7 are not intended to suggest any limitation as to the scope of use or functionality of embodiments of the disclosed subject matter. Neither should the illustrative components be interpreted as having any dependency or requirement related to any single component or combination of components illustrated therein. Additionally, any one or more of the components depicted in any of FIGS. 1-6 may be, in embodiments, integrated with various other components depicted therein (and/or components not illustrated), all of which are considered to be within the ambit of the disclosed subject matter. For example, the lead 700 may be used in the system 100. In addition, the clamp attachment mechanism 712 may be used in connection with leads 200, 300, 400, 500, or 600 as described with reference to FIGS. 1-6.

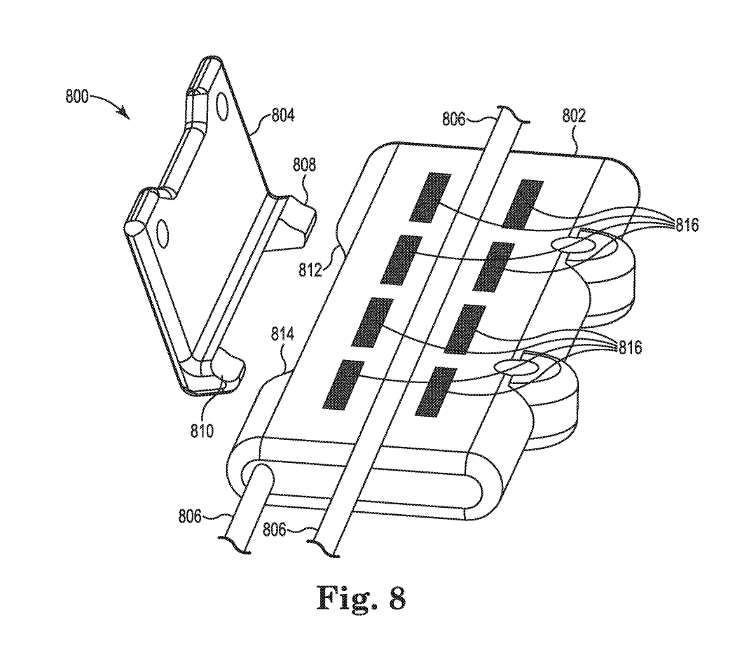

FIG. 8 is an illustration of another example lead 800 that includes attachment mechanisms 808, 810 for at least partially surrounding a target stimulation region in accordance with embodiments of the disclosure. The lead 800 may include a first portion 802, a second portion 804. The lead 800 may also include a two-part lead body 806. As described in further detail above, the lead body 806 may be electrically coupled to at least one electrode 816 arranged with the first portion 802 or the second portion 804. The lead 800 may be configured to at least partially surround the target stimulation region by connecting together the first portion 802 and the second portion 804. In certain instances, the target stimulation region may be an adrenal gland, a peripheral nerve, tissue, or other similar regions that may be difficult to locate and/or difficult to provide targeted stimulation due to the location and/or size of the target region. As shown in FIG. 8, the target stimulation is a nerve 806 arranged between the first portion 802 and the second portion 804.

The first portion 802 and the second portion 804 may include attachment projections 808, 810 and corresponding indentations 812, 814 to facilitate attachment of the first portion 802 to the second portion 804. The attachment projections 808, 810 and the indentations 812, 814 may connect to one another along an exterior section of the first portion 802 and the second portion 804 to connect the first portion 802 and the second portion 804.

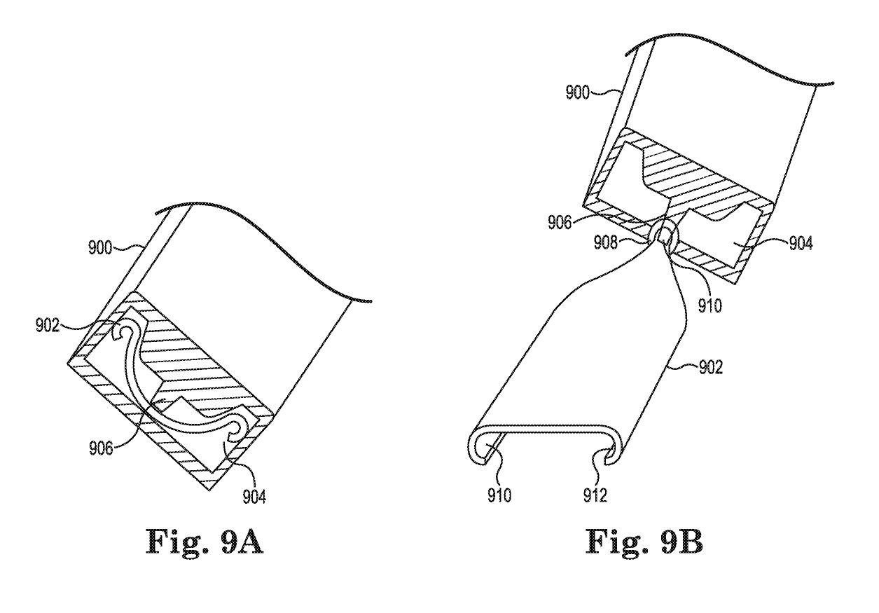

FIG. 9A is an illustration of an example deployment tool 900 and example portion of a lead 902 for at least partially surrounding a target stimulation region in accordance with embodiments of the disclosure. The portion of the lead 902 may be either of a first portion or a second portion of the lead as described above in detail with reference to FIGS. 2-6. The portion of the lead 902 may be arranged within an internal section 904 of the deployment tool 900 in a first configuration and biased in a configuration opposite that of a default configuration of the portion of the lead 902. The deployment tool may include a projection element 906 to assist in biasing the portion of the lead 902.

FIG. 9B is an illustration of the portion of the lead 902, as shown in FIG. 9B, deployed from the deployment tool 900 in accordance with embodiments of the disclosure. After deployment from the deployment tool 900, the portion of the lead 902 resumes the default configuration. As shown in FIG. 9B, the default configuration of the portion of the lead 902 is concave and the biased configuration, shown in FIG. 9A, is convex. The portion of the lead 902 may be deployed (e.g., forced or pushed from) from the internal section 904 of the deployment tool 900 by way of a surgical tool. The portion of the lead 902 may be attached to the deployment tool 900 at an attachment point 910 and may be released from the attachment point by the surgical tool (e.g., forceps or the like). In certain instances, the portion of the lead 902 may be deployed around another portion of a lead for at least partially surrounding a target stimulation. The portion of the lead 902 may include curved sections 910, 912 that surround another portion of a lead (e.g., either a first portion or a second portion as shown in FIGS. 2-6) to connect the portions together.

The illustrative components shown in FIG. 9 are not intended to suggest any limitation as to the scope of use or functionality of embodiments of the disclosed subject matter. Neither should the illustrative components be interpreted as having any dependency or requirement related to any single component or combination of components illustrated therein. Additionally, any one or more of the components depicted in any of FIGS. 1-6 may be, in embodiments, integrated with various other components depicted therein (and/or components not illustrated), all of which are considered to be within the ambit of the disclosed subject matter. For example, the portion of the lead 902 may be used in connection with leads 200, 300, 400, 500, or 600 as described with reference to FIGS. 1-6.

FIG. 10 is a flow chart 1000 of an example method of stimulating a target stimulation region using a lead that includes a first portion and a second portion in accordance with embodiments of the disclosure. The method illustrated in FIG. 10 may use any of the leads described with reference to FIGS. 1-9.

As is shown at block 1002, embodiments of the method include delivering the first portion and the second portion of the lead to the target stimulation region. The delivering the first portion and the second portion may be accomplished by a laparoscopic procedure or other surgical procedure. In certain instances, the first portion and the second portion are separate when delivered to the target stimulation region and in other instances, the first portion and the second portion are connected (e.g., such as those embodiments using a hinge section) when delivered to the target stimulation region.

As is shown at block 1004, embodiments of the method include connecting the first portion to the second portion. In certain instances, connecting the first portion to the second portion occurs via the first aperture and the second aperture to at least partially surround the target stimulation region (e.g., as is described in detail above with reference to FIGS. 1-9). More specifically and in certain instances, connecting the first portion of the second portion via the first aperture and the second aperture includes arranging a threaded section arranged within the first aperture, and arranging a threaded element through the second aperture to engage the threaded element within the threaded section to connect the second portion with the first portion. In other instances, connecting the first portion of the second portion via the first aperture and the second aperture includes arranging a suture through the first aperture and the second aperture to connect the first portion and the second portion.

As is shown at block 1006, the method also may include stimulating the target stimulation region with the at least one electrode. The target stimulation region may include at least one of an adrenal gland, a peripheral nerve, any other type of nerve, and tissue. In addition, the simulation energy may be transmitted to the target stimulation region in a monopolar or multipolar (e.g., bipolar, tripolar, etc.) fashion. In certain instances, pulse generation circuitry may individually control the magnitude of electrical current flowing through each of the electrodes. In these instances, current generators may be used to supply current-regulated amplitudes to selectively generate independent current sources for at least one electrode.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A lead for at least partially surrounding a target stimulation region, the lead comprising:
a first portion including a first aperture;
a second portion including a second aperture;
an attachment element configured to connect the first portion to the second portion via the first aperture and the second aperture to at least partially surround the target stimulation region; and
at least one electrode arranged with one of the first portion and the second portion.

2. The lead of claim 1, wherein the attachment element comprises a threaded element, and further comprising a threaded section arranged within the first aperture, wherein the threaded section is configured to connect with the threaded element arranged through the second aperture to connect the second portion with the first portion.

3. The lead of claim 1, wherein the attachment element comprises aa suture arranged through the first aperture and the second aperture, the suture being configured to connect the second portion with the first portion.

4. The lead of claim 3, wherein an upper surface of the first portion comprises a first area, and a lower surface of the second portion comprises a second area, and the first area is greater than the second area.

5. The lead of claim 4, wherein the at least one electrode comprises a plurality of electrodes, and the second portion covers at least a portion of the plurality of electrodes when the second portion is connected with the first portion.

6. The lead of claim 4, wherein the first portion comprises a first length and a first width, the second portion comprises a second length and a second width, and at least one of the first length is approximately equal to the second length and the first width is approximately equal to the second width.

7. The lead of claim 1, wherein at least one of the first portion and the second portion includes a contoured surface.

8. The lead of claim 7, wherein the first portion comprises the contoured surface, and the at least one electrode is arranged on the first portion, the contoured surface comprising a concave shape, and wherein the concave shape includes a first sloped side and a second sloped side.

9. The lead of claim 8, wherein the at least one electrode comprises a plurality of electrodes, and the plurality of electrodes are arranged on the first sloped side and the second sloped side, and wherein the first sloped side and the second sloped side are configured to direct the electrodes toward a central axis of the first portion.

10. The lead of claim 1, further comprising a hinge section connecting the first portion and the second portion.

11. The lead of claim 1, wherein the target stimulation region comprises at least one of an adrenal gland, a peripheral nerve, and tissue.

12. The lead of claim 1, further comprising a pulse generator, and wherein the lead comprises a lead body, and a proximal portion of the lead body is configured to connect to the pulse generator, and a distal portion of the lead body is configured to attach to at least one of the first portion and the second portion.

13. A lead for at least partially surrounding a target stimulation region, the lead comprising:
a first portion including a first aperture;
a second portion including a second aperture;
an attachment element configured to connect the first portion to the second portion via the first aperture and the second aperture to at least partially surround the target stimulation region;
a hinge section connecting the first portion and the second portion; and
at least one electrode arranged with one of the first portion and the second portion.

14. The lead of claim 13, wherein the first portion, the second portion, and the hinge section include an electrically insulative material.

15. The lead of claim 13, wherein the hinge section is arranged at a first end of the first portion and the second portion, and a second end of the first portion and the second portion comprises the first aperture and the second aperture, and the first aperture and the second aperture are configured to facilitate connection between the first portion and the second portion.

16. The lead of claim 13, wherein at least one of the first portion and the second portion includes a contoured surface.

17. The lead of claim 13, wherein the first portion comprises the contoured surface, and the at least one electrode is arranged on the first portion, the contoured surface comprising a concave shape, wherein the concave shape includes a first sloped side and a second sloped side, the at least one electrode comprises a plurality of electrodes, and the plurality of electrodes are arranged on the first sloped side and the second sloped side.

18. A method of stimulating a target stimulation region using a lead comprising a first portion having a first aperture, a second portion having a second aperture, and at least one electrode arranged with one of the first portion and the second portion, the method comprising:
delivering the first portion and the second portion of the lead to the target stimulation region;
connecting the first portion to the second portion with an attachment element arranged through the first aperture and the second aperture to at least partially surround the target stimulation region; and
stimulating the target stimulation region with the at least one electrode.

19. The method of claim 18, wherein the attachment element comprises a threaded element, and connecting the first portion of the second portion via the first aperture and the second aperture includes arranging a threaded section within the first aperture, and arranging the threaded element through the second aperture to engage the threaded element within the threaded section to connect the second portion with the first portion.

20. The method of claim 18, wherein the attachment element comprises a suture, and connecting the first portion of the second portion via the first aperture and the second aperture includes arranging the suture through the first aperture and the second aperture to connect the first portion and the second portion.
